(19) United States
(12) Patent Application Publication
(10) Pub. No.: US 2020/0164170 A1
(43) Pub. Date: May 28, 2020

(54) Non-Rebreather Mask with Nasal Cannula

(71) Applicant: Bionix Development Corporation, Toledo, OH (US)

(72) Inventors: James Huttner, Sylvania, OH (US); Joshua B. Gaither, Tucson, AZ (US)

(73) Assignee: Bionix Development Corporation, Toledo, OH (US)

(21) Appl. No.: 16/692,061

(22) Filed: Nov. 22, 2019

Related U.S. Application Data
(60) Provisional application No. 62/771,197, filed on Nov. 26, 2018.

(51) Int. Cl. A61M 16/06; A61B 5/08; A61M 16/20

(52) U.S. Cl. CPC A61M 16/0672; A61B 5/082; A61M 16/209; A61M 16/208; A61M 16/06

(57) ABSTRACT

A patient respiratory mask includes a face mask that in use defines an enclosed space, an oxygen reservoir bag in fluid communication with the enclosed space via a one-way intake valve, an out-flow port positioned on the face mask allowing for the exhaust of expired air. A source of oxygen is in fluid communication with the reservoir bag via an oxygen delivery tube, and a nasal cannula is positioned within the enclosed space and is connected to the source of oxygen by a nasal cannula delivery tube that is in fluid communication with the oxygen delivery tube. An in-line flow restrictor is positioned in the nasal cannula delivery tube and allows high-flow oxygen into the reservoir bag, while at the same time limiting oxygen flow into the nasal cannula.

### BACKGROUND OF THE INVENTION

Published research shows that passive oxygenation with high-flow oxygen given via nasal cannula (NC) and a non-rebreather (NRB) mask is more effective for providing patient oxygenation than simple placement of a NRB mask (current standard of care). This combined NC and NRB therapy is currently utilized as the standard of care in both unstable patients undergoing rapid sequence intubation (RSI) and in victims of cardiac arrest.

Currently, there is no single device that provides for both of these functions—high-flow O2 via NC and high flow oxygen via non-rebreather mask. To perform this therapeutic intervention currently requires the provider to first place a nasal cannula, and then secondarily add a non-rebreather mask on top. This takes extra time and creates unnecessary complexity.

### SUMMARY OF THE INVENTION

The current invention describes a non-rebreather mask with a nasal cannula that is suitable to provide high-flow nasal oxygen to reduce the number of components needed to provide this form of therapy and make it a more efficient process.

The non-rebreather mask of the invention includes a face mask that in use defines an enclosed space, an oxygen reservoir bag in fluid communication with the enclosed space via a one-way intake valve, an out-flow port positioned on the face mask allowing for the exhaust of expired air. A source of oxygen is in fluid communication with the reservoir bag via an oxygen delivery tube, and a nasal cannula is positioned within the enclosed space and is connected to the source of oxygen by a nasal cannula delivery tube that is in fluid communication with the oxygen delivery tube. An in-line flow restrictor is positioned in the nasal cannula delivery tube and allows high-flow oxygen into the reservoir bag, while at the same time limiting oxygen flow into the nasal cannula.

### BRIEF DESCRIPTION OF THE DRAWINGS

The advantages of the invention will become readily apparent to those skilled in the art from the following detailed description of various embodiments when considered in the light of the accompanying drawings, in which:

FIG. 1 is a somewhat schematic view of a non-rebreather mask with integrated nasal cannula in accordance with a preferred embodiment of the invention; and

FIG. 2 is a somewhat schematic view of mask of FIG. 1 in use on a patient.

### DETAILED DESCRIPTION OF THE INVENTION

It is to be understood that the specific devices and processes illustrated in the attached drawings and described in the following description are simply exemplary embodiments of the inventive concepts defined in the appended claims. Hence, specific dimensions and other physical characteristics relating to the embodiments disclosed herein should not be considered as limiting, unless the claims expressly state otherwise.

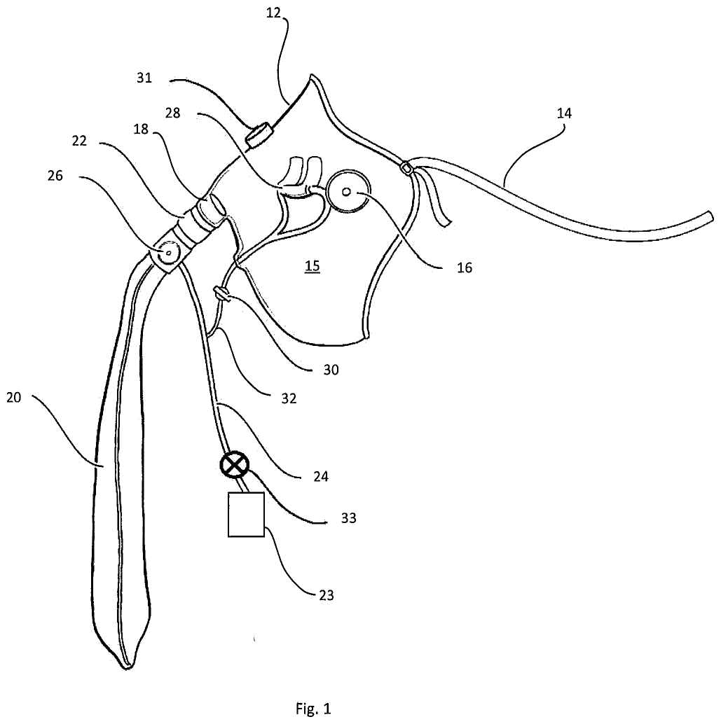

A non-rebreather mask 10 in accordance with a preferred embodiment of the invention is illustrated in FIG. 1. The non-rebreather mask 10 includes a patient face mask 12 that is preferably configured to cover the mouth and nose of a patient. The patient mask 12 is secured to the face of the patient by an elastic band 14 or other conventional securement feature, so that the peripheral edge of the face mask 12 is in a sealing engagement with the face of the patient, defining an enclosed space 15 between the face mask 12 and the face of the patient. A one-way exhalation valve 16 is secured to the face mask 12 and allows for the out-flow of expired air that has been exhaled by the patient into the enclosed space 15.

The face mask 12 also includes a port 18 that provides fluid communication between the enclosed space 15 and an oxygen reservoir bag 20. The reservoir bag 20 is connected to the port 18 via a one-way intake or inhalation valve 22 that allows flow from the oxygen reservoir bag into the enclosed space 15 and prevents flow in the opposite direction. The valve 22 may allow for the inspiration of near 100% fraction of inspired oxygen (FiO₂).

The oxygen reservoir bag 20 is in fluid communication with a source of pure oxygen 23 via an oxygen supply line or delivery tube 24. A pop-off valve 33 located in-line with the oxygen delivery tube 24 prevents overpressure that might result from a blockage. The oxygen delivery tube 24 is preferably connected to the reservoir bag 20 by means of an entrainment valve 26. When open, the entrainment valve 26 admits ambient air as needed to reduce the inspired oxygen (FiO₂) concentration.

In a preferred embodiment, the oxygen delivery tube 24 is also provided with a delivery tube that extends through an opening in the face mask 12, into the enclosed space 15 where the end is connected to and is in fluid communication with a nasal cannula 28 that is preferably sized to be suitable for providing high-flow nasal oxygen. The nasal cannula 28 is preferably integrated with the face mask 12, in that the nasal cannula 28 is preferably rigidly fixed in position relative to the face mask 12 with the distal ends of the cannula extending from the face mask 12 into the enclosed space 15. The nasal cannula 28 may be rigidly fixed to the face mask 12 by any suitable means, such as with an adhesive, or the nasal cannula 28 could be integrally bonded with the face mask 12 to form a monolithic face mask with nasal cannula. A sampling port 31 may be provided for capnography of expired air.

A flow restrictor 30 may be positioned in-line with the nasal cannula delivery tube 32, preferably at a point in the delivery tube 32 outside of the enclosed space 15. In the preferred embodiment illustrated in the drawings, a single oxygen delivery tube 24 supplies both the reservoir bag 20 and the nasal cannula 28, and the flow restrictor 30 may act to reduce the flow to the nasal cannula 28 while at the same time allowing high-flow oxygen to the reservoir bag 20.

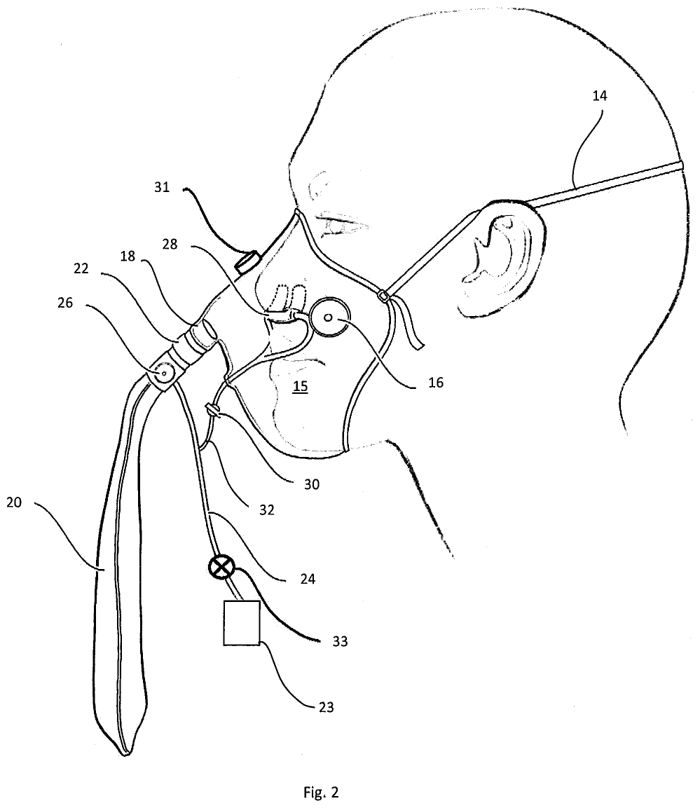

FIG. 2 shows the non-rebreather mask 10 of the preferred embodiment of the invention as it would sit on a patient's face, with the elastic band 14 extending around the head of the patient. The face mask 12 covers both the mouth and the nose of the patient, with the nasal cannula 28 positioned within the nostrils of the patient.

It is understood by those practiced in the art that the shape and design of any patient respiratory mask is primarily dictated by facial anatomy. Accordingly, the shape and design of the current invention will be similar in appearance to currently available rebreather and non-rebreather face masks.

However, the integrated nasal cannula and tubing is sized to allow for administration of high-flow oxygen therapy in accordance with an aspect of the invention. Flow rates may be adjusted according to the intended treatment group. Pre-oxygenation of patients prior to RSI will require lower NC flow rates (ca. 8 l/min) than those patients presenting in full cardiac arrest, for whom a much higher oxygen NC flow rate (ca. 15 l/min) may be used. The NRB flow rate is generally set to ca. 20 l/min (flush rate). It is understood that oxygen flow rates of over ca. 15 l/min will need humidification to be tolerated by the patient and avoid nasal irritation.

It is intended that the high-flow nasal cannula and the oxygen reservoir bag will be fed by the same, single oxygen tank or reservoir. Because the oxygen flow and pressure to the nasal cannula and the reservoir bag can be significantly different (e.g. 8-15 l/min for the nasal cannula vs 15-25 l/min for the non-rebreather reservoir bag), it will be necessary to have a valve or other flow restrictive device in-line with the nasal cannula. It is understood by those practiced in the art that many means may be used to restrict or limit air flow, including valves, different sized tubing, and other types of flow-restrictors.

### Clinical Use of the Invention

There are two primary patient populations in which this device may be used: victims of cardiac arrest (both traumatic and non-traumatic) and unstable patients undergoing rapid sequence intubation (RSI).

For use in victims of cardiac arrest, upon arrival at a cardiac arrest scene, the provider(s) will place the proposed mask on the victim, assuring that the nasal prongs are inserted into the flares, and otherwise securing the mask onto the patient using elastic straps in the usual fashion. Oxygen will then be connected to the mask tubing, and the flow will be adjusted to 15 l/min (or other high-flow rate as dictated by medical protocol) by the nasal cannula and the in-line flow-restrictor, while oxygen can still be delivered into the reservoir bag at a higher rate, for example, of 15-25 l/min.

During the initial resuscitation effort, the high-flow oxygen provided by the integrated nasal cannula will enhance patient oxygenation through dead-space wash-out, and avoid interruption of chest compressions, thereby enhancing the patient's chance of survival. If the resuscitation effort is successful and the patient resumes respirations, the non-rebreather oxygen reservoir will provide the patient near 100% FiO2 during transport to the hospital.

For use in unstable patients undergoing RSI the mask will be placed in the same manner as described above and removed once the patient has been completely paralyzed and just prior to intubation.

In accordance with the provisions of the patent statutes, the invention has been described in what is considered to represent its preferred embodiments. However, it should be noted that the invention could be practiced otherwise than as specifically illustrated and described without departing from its spirit or scope.

What is claimed is:

1. A patient respiratory mask comprising:
a face mask that in use defines an enclosed space between an inner surface of the face mask and the face of a patient;
an oxygen reservoir bag in fluid communication with the enclosed space via a one-way intake valve, the one-way intake valve allowing flow from the reservoir bag into the enclosed space but preventing the entry of expired air into the reservoir bag;
an out-flow port positioned on the face mask allowing for the exhaust of expired air;
a source of oxygen in fluid communication with the reservoir bag via an oxygen delivery tube;
a nasal cannula sized for high-flow oxygen delivery positioned within the enclosed space defined by the face mask, the nasal cannula being connected to the source of oxygen by a nasal cannula delivery tube that is in fluid communication with the oxygen delivery tube; and
an in-line flow restrictor positioned in the nasal cannula delivery tube that limits oxygen flow into the nasal cannula, while at the same time allowing a higher flow of oxygen into the reservoir bag.

2. The patient respiratory mask of claim 1, wherein the out-flow port includes a one-way valve to provide the effective near 100% FiO₂ of a non-rebreather mask by preventing inspiration of room air.

3. The patient respiratory mask of claim 1, wherein there is an in-line pop-off valve in the oxygen delivery tube, positioned between the source of oxygen and the connection of the nasal cannula delivery tube with the oxygen delivery tube, to prevent overpressure of the system.

4. The patient respiratory mask of claim 1, further comprising a sampling port in the face mask that allows for capnography of expired air.

5. The patient respiratory mask of claim 1, wherein the nasal cannula is formed integrally with the face mask.

6. The patient respiratory mask of claim 1, wherein the flow restrictor allows the flow of oxygen into the nasal cannula at a rate of about 8 to 15 l/min, while at the same time allowing oxygen flow into the reservoir bag at a flow rate of about 15-25 l/min.

7. A method of administering high-flow oxygen therapy to a patient utilizing the respiratory mask of claim 1.
